(19) United States
(12) Patent Application Publication
(10) Pub. No.: US 2020/0317822 A1
(43) Pub. Date: Oct. 8, 2020

(54) Method for Preparing Arabinogalacturonan from Tangerine Peel

(71) Applicant: SOUTH CHINA BOTANICAL GARDEN, CHINESE ACADEMY OF SCIENCES, Guangzhou (CN)

(72) Inventor: Bao YANG, Guangzhou (CN)

(73) Assignee: SOUTH CHINA BOTANICAL GARDEN, CHINESE ACADEMY OF SCIENCES, Guangzhou (CN)

(21) Appl. No.: 16/304,142

(22) PCT Filed: Apr. 20, 2018
(86) PCT No.: PCT/CN2018/083829

§ 371 Date: Nov. 22, 2018

(30) Foreign Application Priority Data
Mar. 26, 2018 (CN) 201810254034.1

(51) Int. Cl. C08B 37/00; B01D 11/02; B01D 15/36; B01D 15/34; B01D 15/42

(52) U.S. Cl. CPC C08B 37/0003; C08B 37/006; B01D 15/426; B01D 15/363; B01D 15/34; B01D 11/0288

(57) ABSTRACT

A method for preparing an arabinogalacturonan, including the following steps. Grinding a tangerine peel and performing an extraction with water; separating and collecting an extract solution, concentrating the extract solution and adding ethanol to precipitate crude tangerine peel polysaccharides, and collecting the crude tangerine peel polysaccharides; purifying the crude tangerine peel polysaccharides over an anion exchange column, gradient eluting with water and NaCl solution, and collecting a fraction eluted with the NaCl solution; concentrating the fraction eluted with the NaCl solution, then purifying over a gel filtration column, eluting with water, and collecting a target fraction; and concentrating and drying the target fraction to obtain the arabinogalacturonan. The method achieves the preparation of arabinogalacturonan from the tangerine peel, with a yield of 6-45 g/kg and a purity of 80%-95%.

### CROSS REFERENCE TO RELATED APPLICATIONS

This application is the national phase entry of International Application No. PCT/CN2018/083829, filed on Apr. 20, 2018, which is based upon and claims priority to Chinese Application No. CN201810254034.1, filed on Mar. 26, 2018, the entire contents of which are incorporated herein by reference.

### TECHNICAL FIELD

The present invention relates to the technical field of agricultural product processing, and particularly relates to a method for preparing arabinogalacturonan from tangerine peel.

### BACKGROUND

Tangerine peel, the mature peel of tangerine (Citrus reticulata Blanco) which belongs to Rutaceae family and Citrus genus, is mainly produced in several provinces in southern China. Tangerine peel is an important traditional Chinese medicinal material, serving as both medicine and food. Tangerine peel can be used to treat vomiting, improve gastrointestinal function and promote digestion. Tangerine peel has a good efficacy in treating cardiovascular diseases and can effectively lower blood lipids and blood pressure. Tangerine peel also shows antibacterial, anti-inflammatory, expectorant and anti-asthmatic effects. For cooking and seasoning, tangerine peel can be used to remove fishy smell and improve food flavor. At present, reports on the active substances in tangerine peel focus on aromatic essential oils and methoxyflavonoids, while structural characteristics of the major polysaccharide components therein have not yet been identified. Thus, there is a need to carry out research on separation, purification and structural identification of polysaccharides in tangerine peel so as to reveal the structures of major polysaccharides, which is of great significance for clarifying the chemical compositions of tangerine peel.

### SUMMARY

One objective of the present invention is to provide a method for preparing arabinogalacturonan from tangerine peel.

The inventor has discovered and prepared one major polysaccharide in tangerine peel, which is identified by NMR analysis to be arabinogalacturonan, having a chemical structure as formula (I):

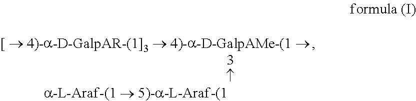

wherein R represents hydrogen or methyl ester group.

In general, “GalpA” in the formula (I) is an abbreviation for “galacturonic acid”, and “Araf” refers to arabinose.

The method for preparing arabinogalacturonan, of the present invention, comprises the following steps:

grinding tangerine peel and extracting with water; separating and collecting an extract solution, concentrating the extract solution and adding ethanol into the concentrated extract solution to precipitate crude tangerine peel polysaccharides, and collecting the crude tangerine peel polysaccharides; purifying the crude tangerine peel polysaccharides over an anion exchange column, gradient eluting with water and NaCl solution, and collecting a fraction eluted with NaCl solution; concentrating the fraction eluted with NaCl solution, purifying over a gel filtration column, eluting with water, and collecting a target fraction; concentrating and drying the target fraction to obtain arabinogalacturonan.

Preferably, the steps of “purifying the crude tangerine peel polysaccharides over an anion exchange column, gradient eluting with water and NaCl solution, and collecting a fraction eluted with NaCl solution; concentrating the fraction eluted with NaCl solution, purifying over an gel filtration column, eluting with water, and collecting a target fraction; concentrating and drying the target fraction to obtain arabinogalacturonan” comprise: purifying the crude tangerine peel polysaccharides over the anion exchange column, eluting with water to remove neutral polysaccharide fractions, then eluting with NaCl solution, and collecting the fraction eluted with NaCl solution; concentrating the fraction eluted with NaCl solution, purifying over the gel filtration column, eluting with water as a solvent, and collecting a target polysaccharide fraction; concentrating and drying the target polysaccharide fraction to obtain arabinogalacturonan.

Preferably, the step of grinding tangerine peel and extracting with water comprises: adding the ground tangerine peel into water wherein a weight of the water is 1-30 times that of the ground tangerine peel, and extracting at 25° C.-100° C. for 1-120 hours.

Preferably, the step of separating and collecting an extract solution comprises: separating by centrifugation or filtration.

Preferably, the step of adding ethanol into the concentrated extract solution to precipitate crude tangerine peel polysaccharides comprises: adding ethanol until a final volume fraction of the extract solution is 20%-90%.

Preferably, the tangerine peel is fresh tangerine peel or dried tangerine peel.

Preferably, the NaCl solution is 0.2 M NaCl aqueous solution.

The present invention further provides the use of tangerine peel in preparing arabinogalacturonan.

The arabinogalacturonan prepared by the present invention shows a high immunopotentiating activity.

The present invention achieves the preparation of arabinogalacturonan from tangerine peel, with a yield of 6-45 g/kg and a purity of 80%-95%. The present invention has introduced a novel method for preparing arabinogalacturonan. It is helpful for extensive utilization of tangerine peel and improving the additional value of tangerine peel products, which is of great significance for promoting the sustainable development of the industry.

### DETAILED DESCRIPTION OF THE EMBODIMENTS

The following embodiments are used for further describing this invention rather than limiting the invention.

### Embodiment 1: Preparation and Structure Identification of Arabinogalacturonan

1. Preparation of Arabinogalacturonan

1) Material: Fresh tangerine peels were collected and ground.

2) Extraction: The tangerine peels were added into water wherein a weight of the water was 15 times that of the tangerine peels. The extraction was conducted at 50° C. for 2 hours. After centrifugation, the extract solution was collected.

3) Solvent precipitation: The extract solution was concentrated and ethanol was added to a final volume fraction of 60% (extract solution/ethanol=60/40, v/v). After incubation at 4° C. for 12 hours, the pellets were collected by centrifugation. The pellets were crude tangerine peel polysaccharides.

4) Column purification: The crude tangerine peel polysaccharides were purified over a DEAE Sepharose Fast Flow anion exchange column which was eluted with water and subsequently with NaCl solution (0.2 M). The fraction eluted with NaCl solution was collected, concentrated and then purified over a Sephadex G100 gel filtration column (15×460 mm) which was eluted with water at a flow rate of 0.5 ml/min. Polysaccharide fraction eluted at 50-160 min was collected. The fraction was then concentrated and dried and thereby a polysaccharide A (arabinogalacturonan) was obtained.

The yield of arabinogalacturonan prepared by this protocol was 26-35 g/kg, with a purity of 80%-95%.

2. Structure Identification of the Polysaccharide a (Arabinogalacturonan)

The polysaccharide A (arabinogalacturonan) is water soluble. Results of ¹H NMR (500 MHz, D₂O) and ¹³C NMR (125 MHz) analysis are shown in Table 1.

TABLE 1
¹³C and ¹H chemical shifts of arabinogalacturonan from tangerine peel

| Glycosidic bond | Residue | | 1 | 2 | 3 | 4 | 5 | 6 |
|---|---|---|---|---|---|---|---|---|
| α-L-Araf-(1→ | A | C | 107.8 | 81.6 | 77.1 | 84.5 | 61.7 | |
| | | H | 5.08 | 4.13 | 3.96 | 4.04 | 3.70, | |
| | | | | | | | 3.81 | |
| →5)-α-L-Araf-(1→ | B | C | 107.6 | 81.7 | 77.3 | 82.9 | 67.0 | |
| | | H | 5.15 | 4.13 | 4.00 | 4.21 | 3.79, | |
| | | | | | | | 3.88 | |
| →4)-α-D-GalpAMe- | C | C | 100.9 | 68.5 | 68.5 | 79.2 | 71.2 | 171.3 |
| (1→ ^{a} | | H | 4.95 | 3.68 | 4.02 | 4.47 | 5.08 | |
| →4)-α-D-GalpA- | D | C | 99.9 | 68.5 | 69.0 | 78.3 | 72.1 | 175.7 |
| (1→ | | H | 5.10 | 3.69 | 3.93 | 4.42 | 4.72 | |
| →3,4)-α-D- | E | C | 100.9 | 68.4 | 79.1 | 79.2 | 71.1 | 171.4 |
| GalpAMe(1→ | | H | 4.95 | 3.74 | 4.35 | 4.47 | 5.14 | |

According to the NMR results, the polysaccharide A is identified as arabinogalacturonan. The structure is shown in formula (I).

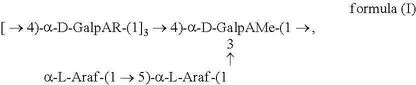

wherein R represents hydrogen or methyl ester group.

### Embodiment 2

1) Material: Fresh tangerine peels were collected and ground.

2) Extraction: The tangerine peels were added into water wherein a weight of the water was 1 time that of the tangerine peels. The extraction was conducted at room temperature (25° C.) for 120 hours. After filtration, the extract solution was collected.

3) Solvent precipitation: The extract solution was concentrated and ethanol was added to a final volume fraction of 50% (extract solution/ethanol=50/50, v/v). After incubation at 4° C. for 12 hours, the pellets were collected by centrifugation. The pellets were crude tangerine peel polysaccharides.

4) Column purification: The crude tangerine peel polysaccharides were purified over a DEAE Sepharose Fast Flow anion exchange column which was eluted with water and subsequently with NaCl solution (0.2 M). The fraction eluted with NaCl solution was collected, concentrated and then purified over a Sephadex G100 gel filtration column which was eluted with water. The target polysaccharide fraction was collected. The fraction was then concentrated and dried and thereby a polysaccharide A (arabinogalacturonan) was obtained. Structure identification showed the same result with embodiment 1

The yield of arabinogalacturonan prepared by this protocol was 6-16 g/kg, with a purity of 80%-95%.

### Embodiment 3

1) Material: Fresh tangerine peels were collected and ground.

2) Extraction: The tangerine peels were added into water wherein a weight of the water was 30 times that of the tangerine peels. The extraction was conducted at 100° C. for 5 hours. After filtration, the extract solution was collected.

3) Solvent precipitation: The extract solution was concentrated and ethanol was added to a final volume fraction of 90% (extract solution/ethanol=90/10, v/v). After incubation at 4° C. for 12 hours, the pellets were collected by centrifugation. The pellets were crude tangerine peel polysaccharides.

4) Column purification: The crude tangerine peel polysaccharides were purified over a DEAE Sepharose Fast Flow anion exchange column which was eluted with water and subsequently with NaCl solution (0.2 M). The fraction eluted with NaCl solution was collected, concentrated and then purified over a Sephadex G100 gel filtration column which was eluted with water. The target polysaccharide fraction was collected. The fraction was then concentrated and dried and thereby a polysaccharide A (arabinogalacturonan) was obtained. Structure identification showed the same result with embodiment 1

The yield of arabinogalacturonan prepared by this protocol was 32-45 g/kg, with a purity of 80%-95%.

### Embodiment 4

1) Material: Dried tangerine peels were collected and ground.

2) Extraction: The tangerine peels were added into water wherein a weight of the water was 30 times that of the tangerine peels. The extraction was conducted at 60° C. for 1 hour. After filtration, the extract solution was collected.

3) Solvent precipitation: The extract solution was concentrated and ethanol was added to a final volume fraction of 20% (extract solution/ethanol=20/80, v/v). After incubation at 4° C. for 12 hours, the pellets were collected by centrifugation. The pellets were crude tangerine peel polysaccharides.

4) Column purification: The crude tangerine peel polysaccharides were purified over a DEAE Sepharose Fast Flow anion exchange column which was eluted with water and subsequently with NaCl solution (0.2 M). The fraction eluted with NaCl solution was collected, concentrated and then purified over a Sephadex G100 gel filtration column which was eluted with water. The target polysaccharide fraction was collected. The fraction was then concentrated and dried and thereby a polysaccharide A (arabinogalacturonan) was obtained. Structure identification showed the same result with embodiment 1

The yield of arabinogalacturonan prepared by this protocol was 11-23 g/kg, with a purity of 80%-95%.

What is claimed is:

1. (canceled)

2. A method for preparing an arabinogalacturonan, comprising isolating the arabinogalacturonan from a tangerine peel; and the arabinogalacturonan has a chemical structure shown as formula (I): 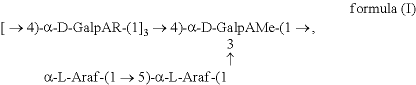
wherein R represents a hydrogen or a methyl ester group.

3. The method of claim 2, comprising the following steps:
grinding the tangerine peel and performing an extraction with water;
separating and collecting an extract solution, concentrating the extract solution to obtain an concentrated extract solution, and adding ethanol into the concentrated extract solution to precipitate crude tangerine peel polysaccharides, and collecting the crude tangerine peel polysaccharides;
purifying the crude tangerine peel polysaccharides over an anion exchange column, gradient eluting with water and NaCl solution, and collecting a fraction eluted with the NaCl solution;
concentrating the fraction eluted with the NaCl solution, then purifying over a gel filtration column, eluting with water as a solvent, and collecting a target polysaccharide fraction; and
concentrating and drying the target polysaccharide fraction to obtain the arabinogalacturonan.

4. The method of claim 3, wherein the step of gradient eluting with the water and the NaCl solution comprises: gradient eluting with the water to remove neutral polysaccharide fractions, then eluting with the NaCl solution.

5. The method of claim 3, wherein the step of grinding the tangerine peel and performing the extraction with the water comprises: adding a ground tangerine peel into the water; wherein a weight of the water is 1-30 times that of the ground tangerine peel, and the extraction are performed at 25° C.-100° C. for 1-120 hours.

6. The method of claim 3, wherein the step of separating and collecting the extract solution comprises: separating the extract solution by means of a centrifugation or a filtration.

7. The method of claim 3, wherein the step of adding the ethanol into the concentrated extract solution to precipitate the crude tangerine peel polysaccharides comprises: adding the ethanol until a final volume fraction of the concentrated extract solution is 20%-90%.

8. The method of claim 3, wherein the tangerine peel is a fresh tangerine peel or a dried tangerine peel.

9. The method of claim 3, wherein the NaCl solution is 0.2 M NaCl aqueous solution.

10. (canceled)
